(19) United States
(12) Patent Application Publication
(10) Pub. No.: US 2025/0057411 A1
(43) Pub. Date: Feb. 20, 2025

(54) SYSTEMS, DEVICES, AND METHODS FOR IMPLEMENTING COMPUTERIZED AMSLER GRID FOR EVALUATION OF VISUAL DISTURBANCES

(71) Applicants: Stephane Krumenacker, New York, NY (US); Thomas Jefferson University, Philadelphia, PA (US)

(72) Inventors: James J. Evans, Newtown Square, PA (US); Nikolaos Mouchtouris, Philadelphia, PA (US); Vadim Geyfman, Marlton, NJ (US); Stephane Krumenacker, New York, NY (US)

(21) Appl. No.: 18/723,103

(22) PCT Filed: Dec. 22, 2022
(86) PCT No.: PCT/US22/53873

§ 371 Date: Jun. 21, 2024

Related U.S. Application Data
(60) Provisional application No. 63/293,263, filed on Dec. 23, 2021.

(51) Int. Cl. A61B 3/024; A61B 3/00; G16H 15/00

(52) U.S. Cl. CPC A61B 3/024; A61B 3/0033; A61B 3/0058; G16H 15/00

(57) ABSTRACT

A computer-implemented method is provided herein. One aspect of the method provides a computer-implemented method including: displaying a neuro-ophthalmic examination pattern comprising a plurality of segments, wherein each of the plurality of segments is displayed at a time offset and at a discrete location of a display screen compared to other segments of the plurality of segments; receiving user input during a time period which a given segment of the plurality of segments is displayed on the display screen; and generating a neuro-ophthalmic examination report based on the displaying the neuro-ophthalmic examination pattern and the received user input.

### CROSS-REFERENCE TO RELATED APPLICATION

The present application claims priority to U.S. Provisional Patent Application No. 63/293,263, filed Dec. 23, 2021, which is incorporated herein by reference in its entirety.

### BACKGROUND

Conventional medical practices are often limited to in-person meetings between a patient and a medical professional. This can be a great burden on a patient, particularly where the patient lives a significant distance away from a corresponding medical center, or if the patient's medical condition requires numerous patient-medical professional interactions.

Telemedicine offers the ability to reduce these patient burdens. However, while advances have been made in telemedicine, conventional telemedicine platforms are limited in their ability to perform certain examinations.

### SUMMARY

One aspect of the invention provides a computer-implemented method including: displaying a neuro-ophthalmic examination pattern comprising a plurality of segments, wherein each of the plurality of segments is displayed at a time offset and at a discrete location of a display screen compared to other segments of the plurality of segments; receiving user input during a time period which a given segment of the plurality of segments is displayed on the display screen; and generating a neuro-ophthalmic examination report based on the displaying the neuro-ophthalmic examination pattern and the received user input.

Another aspect of the invention provides a device for generating a neuro-ophthalmic examination report. The device includes a display screen. The device also includes a user input mechanism. The device also includes one or more processors configured to execute a set of instructions that cause the one or more processors to: (a) display, via the display screen, a neuro-ophthalmic examination pattern comprising a plurality of segments, wherein each of the plurality of segments is displayed at a time offset and at a discrete location of a display screen compared to other segments of the plurality of segments; (b) receive, via the user input mechanism, user input during a time period which a given segment of the plurality of segments is displayed on the display screen; and (c) generate a neuro-ophthalmic examination report based on the displaying the neuro-ophthalmic examination pattern and the received user input.

Another aspect of the invention provides a computer-readable medium for generating a neuro-ophthalmic examination report. The computer-readable medium includes one or more processors. The computer-readable medium also includes memory. The computer-readable medium also includes a set of instructions stored in the memory that, when executed by the one or more processors, cause the one or more processors to: (a) display, via a display screen, a neuro-ophthalmic examination pattern comprising a plurality of segments, wherein each of the plurality of segments is displayed at a time offset and at a discrete location of a display screen compared to other segments of the plurality of segments; (b) receive user input during a time period which a given segment of the plurality of segments is displayed on the display screen; and (c) generate a neuro-ophthalmic examination report based on the displaying the neuro-ophthalmic examination pattern and the received user input.

### BRIEF DESCRIPTION OF THE DRAWINGS

For a fuller understanding of the nature and desired objects of the present invention, reference is made to the following detailed description taken in conjunction with the accompanying drawing figures wherein like reference characters denote corresponding parts throughout the several views.

FIG. 1 depicts a system for generating a neuro-ophthalmic examination according to an embodiment of the present disclosure.

FIG. 2 depicts a server for generating a neuro-ophthalmic examination according to an embodiment of the present disclosure.

FIGS. 3-9 depict visual segments of a neuro-ophthalmic examination according to an embodiment of the present disclosure.

FIG. 10 depicts a process flow for generating a neuro-ophthalmic examination according to an embodiment of the present disclosure.

FIGS. 11A-11D depict visual segments of a neuro-ophthalmic examination according to an embodiment of the present disclosure.

FIGS. 12A-12B depict visual segments of a neuro-ophthalmic examination according to an embodiment of the present disclosure.

FIGS. 13A-13B depict visual segments of a neuro-ophthalmic examination according to an embodiment of the present disclosure.

### DEFINITIONS

The instant invention is most clearly understood with reference to the following definitions.

As used herein, the singular form “a,” “an,” and “the” include plural references unless the context clearly dictates otherwise.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

Unless specifically stated or obvious from context, the term “or,” as used herein, is understood to be inclusive.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

### DETAILED DESCRIPTION OF THE INVENTION

A traditional Amsler grid is a 10 cm by 10 cm grid that consists of vertical and horizontal lines that create 0.5 cm squares with a fixation marker in the center of the grid. This assessment is used by physicians to detect any visual disturbances in the user's peripheral vision that would be concerning for a number of diseases, such as macular degeneration, glaucoma, macular edema, chorioretinopathy, optic neuritis, ocular hypertension, optic neuropathy, and the like. The user (e.g., a patient) is instructed to close one eye and use the other eye to focus on a dot in the middle of the grid. The user is then instructed to identify if any of the squares in the periphery appear distorted.

When a traditional Amsler grid assessment is administered via a computer, identifying the distorted squares can be problematic. Computerized Amsler grids typically require the user to click, via a mouse, the particular location that appears distorted. However, the squares that appear distorted when one focuses on the central dot no longer appear distorted when the user tries to click on them, because the user naturally shifts his or her focus onto the mouse pointer and where the mouse pointer is on the Amsler grid. The distorted area of interest ceases to be in the user's peripheral vision when the user tries to click on it, so a user may inaccurately indicate regions of the Amsler grid that appear distorted.

Embodiments of the present disclosure provide a computerized version of the Amsler grid that can be performed on any computer without the need for a touch screen or specialized goggles. Segments of an Amsler grid may be displayed on an electronic display in a piecewise fashion. For example, in a particular embodiment, the squares of the grid are displayed in an automated, clockwise fashion while the user is focused on the central fixation marker. Once a set of squares appears distorted, the user presses a keyboard key to indicate the presence of the visual disturbances. The location of the visual disturbance on the grid is saved. The size of the grid, the color of the vertical and horizontal lines that make up the grid, the speed of the automated clockwise display of squares can all be modified to adjust the sensitivity/specificity of the assessment. To improve accuracy of user responses, the Amsler grid can be displayed multiple times in different ways: the squares of the grid can be displayed in clockwise, counterclockwise fashion or in random order until the entire grid is filled. This improves test-retest reliability.

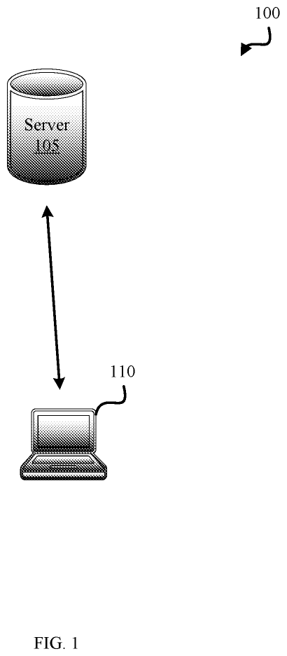

FIG. 1 depicts a system for generating a neuro-ophthalmic examination according to an embodiment of the present disclosure. The system can include a server 105 and a computing device 110.

The server 105 can store instructions for generating a neuro-ophthalmic examination, for example, a segmented Amsler grid. In some cases, the server 105 can also include a set of processors that execute the set of instructions. Further, the server 105 can be any type of server capable of storing and/or executing instructions, for example, an application server, a web server, a proxy server, a file transfer protocol (FTP) server, and the like. In some cases, the server 105 can be a part of a cloud computing architecture, such as a Software as a Service (SaaS), Development as a Service (DaaS), Data as a Service (DaaS), Platform as a Service (PaaS), and Infrastructure as a Service (IaaS).

A computing device 110 can be in electronic communication with the server 105 and can display the neuro-ophthalmic examination to a user. The computing device 110 can include a display for displaying the neuro-ophthalmic examination, and a user input device, such as a mouse, keyboard, or touchpad, for logging and transmitting user input corresponding to the neuro-ophthalmic examination. In some cases, the computing device 110 can include a set of processors for executing the neuro-ophthalmic examination (e.g., from instructions stored in memory). Examples of a computing device include, but are not limited to, a personal computer, a laptop, a tablet, a cellphone, a personal digital assistant, an e-reader, a mobile gaming device, and the like.

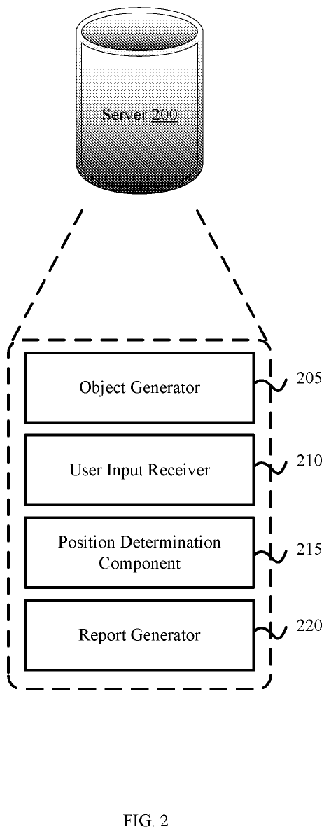

FIG. 2 depicts a server 200 for generating a neuro-ophthalmic examination according to an embodiment of the present disclosure. The server can be an example of the server 105 as discussed with reference to FIG. 1. The server 200 can include an object generator 205, a user input receiver 210, an object position determination component 215, and a report generator 220.

The object generator 205 can generate visual segments of the neuro-ophthalmic examination for the display screen of the computing device of a user, such as the computing device 110 as described with reference to FIG. 1. The visual segment can be any number of objects having a defined body, which includes but is not limited to a dot, a circle, a triangle, a star, a rectangle, an ellipse, and the like. However, examples below implement the embodiment of where the visual segments form a grid segment, which when compiled together, the plurality of visual segments form an Amsler grid. Thus, each visual segment may form a set of “squares” that form a section of an Amsler grid. Further, the size of each visual segment may be variable. For example, in the case of an Amsler grid, each visual segment may be as small as a single “square” of the Amsler grid, or may be 2 “squares”, 5 “squares”, an eighth of an Amsler grid, a quarter of an Amsler grid, and the like. Further, a size of a visual segment may be variable amongst the plurality of visual segments. For example, one visual segment may be 2 “squares” of an Amsler grid, and another visual segment may be 3 “squares”, and the like. In some other cases, the size of the visual segments may be selected based on an field of view angle range the examination is intended to cover. For example, the larger the individual squares of an Amsler grid are, the larger the field of vision the Amsler grid may test for a user (e.g., 15 degrees, 20, degrees, 30 degrees from center, and the like).

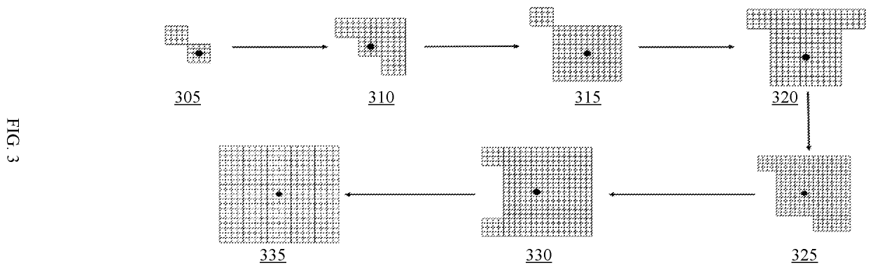

The object generator 205 can generate each visual segment at a different location and at a different time period with respect to the other visual segments to be/have been displayed. For example, FIG. 3 depicts a timespan of displaying a segmented Amsler grid according to an embodiment of the present disclosure. The display, such as the display of the computing device 110, may initially display two sections of the grid at step 305. At step 310, the display can display additional sections of the Amsler grid, where each additional section is displayed at a time offset compared to one another. Eventually, the display can display, steps 315, 320, 325, and 330, until the display ends with a full Amsler grid at step 335. In some cases, as the display displays a subsequent visual segment, the display can remove the preceding segment, such that a user sees only a single (or other subset) of the visual segments at one time. In other cases, the display may continue to display the preceding visual segments, such that the display may ultimately display the entire aggregated pattern (e.g., a full Amsler grid as in FIG. 4).

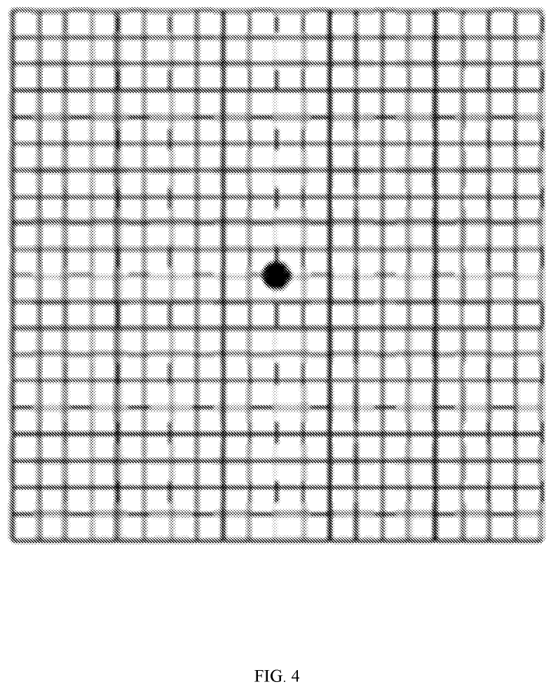

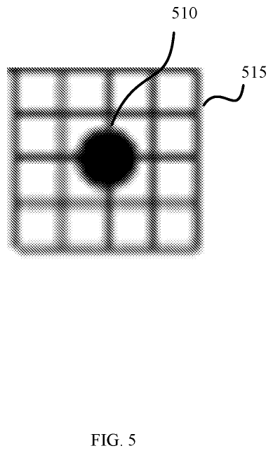

Following the example where the fully displayed pattern is an Amsler grid, the object generator 205 may generate a visual segment with an additional segment as part of the visual segment, for example a center dot 510 in visual segment 515 of FIG. 5. In these cases, the system (e.g., system 100) may instruct a user to focus his or her vision on the center dot 510 as the display displays the other visual segments (not shown in FIG. 5) over time (e.g., every 3 seconds, every 5 seconds, every 10 seconds, every 15 seconds, and the like). The system may also instruct the user to provide input when the user views a distorted version of a displayed visual segment.

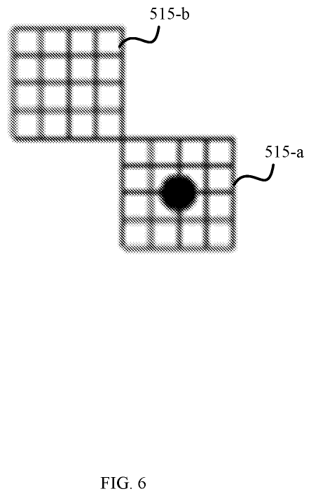

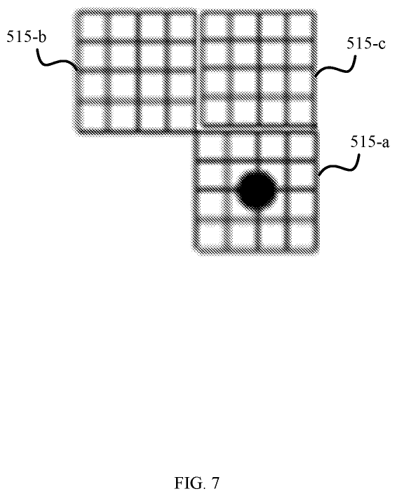

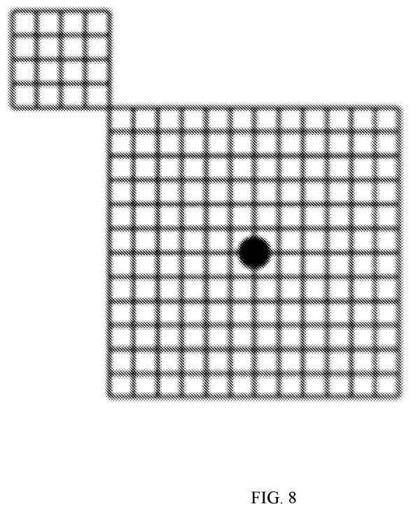

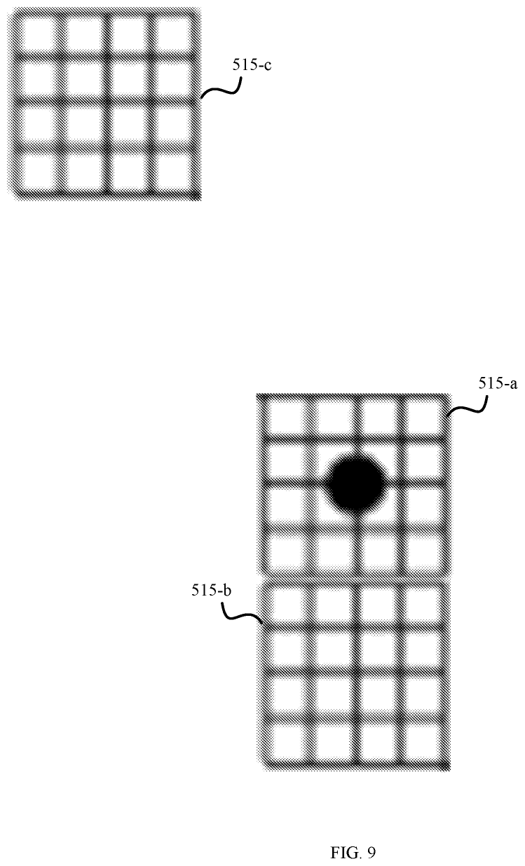

The display pattern may be displayed in some cases according to a predetermined pattern. For example, the predetermined pattern may be a “clockwise” pattern, where the visual segments over time are displayed clockwise in relation to a particular point of the display (e.g., a center point). Likewise, in some cases, the predetermined pattern may be a “counter-clockwise” pattern. In some cases, the predetermined pattern may be an outwardly spanning pattern, such as those depicted in FIGS. 6-8. In FIG. 6, visual segments 515-a and 515-b may be initially displayed. In FIG. 7, an additional time period may begin corresponding to visual segment 515-c. The display may then display the visual segment 515-c (e.g., either with or without segments 515-a, 515-b). At FIG. 8, the display may continue to display additional visual segments until a particular perimeter around the center is filled, at which point the display will begin displaying visual segments in the next outer perimeter with respect to the center point. Other predetermined patterns may be implemented as well. Alternatively, the object generator 205 may generate the visual segments in a random fashion, such that the positions of the visual segments (515-a, b, and c of FIG. 9) are randomly selected.

The user input receiver 210 can receive user input from the computing device. For example, the user input can be a mouse click, a keyboard click, a touch on a touchpad, and the like. The user input receiver 210 can receive the user input and log different parameters of the user input. For example, the user input receiver 210 can identify a timestamp of the user input, the type of user input (e.g., mouse click, keyboard click, etc.) and the like. The server 200 may store the user input in memory.

The position determination component 215 can determine an identity of the most recently displayed visual segment based on the received user input. The position determination component 215 can determine the time the user provides input via the computing device. The determination can be based on a timestamp of the received user input, for example. From the determined time, the position determination component 215 can determine which of the visual segment is being displayed on the display at that time. In cases where multiple visual segments are displayed at one time, the position determination component 215 can determine the most recently displayed visual segment based on the timing of the user input. In some cases, the determination can be based on a predefined speed, a predefined display pattern, and the like, for determining the identity of the visual segment.

The report generator 220 can determine a neuro-ophthalmic report for a user based on the received user input and the displayed pattern. For example, in the case of a displayed Amsler grid, the report generator can determine that a user of the computing device display experiences peripheral distortion in a particular location of the user's field of vision. The report generator 220 can make this determination according to the identity of the visual segment corresponding to received user input. The report generator 220 can also make this determination based on a given display size of the visual segment. For example, a smaller visual segment may correspond to a smaller portion of the user's field of vision as opposed to a larger visual segment.

In some cases, the report generator 220 can determine an angle from center which the vision distortion is experienced. For example, the system can determine a distance away from the display the user is positioned, such as by performing a blind spot detection procedure. The blind spot detection procedure can reposition an object across the display screen. The user may be instructed to provide user input (e.g., via a mouse, keyboard, and the like) when the repositionable object is located in an area where the user cannot see the object (e.g., while the user is focused on a particular static object of the display screen). The user may also be instructed to provide input once the object reappears in the user's vision. From the length of the blind spot experienced by the user, the system can determine how far away the user is positioned from the screen. Based on the determined distance and the position of the visual segment corresponding to user input, the report generator 220 can determine an angle from center where the vision distortion is experienced.

As the user may indicate multiple visual segments where a visual distortion is experienced, the report generator 220 may generate a report of one or more positions (e.g., angles from center) where the user experienced visual distortions. The visual distortion locations for a user may be indicative of, or correspond to, various neuro-ophthalmic conditions, such as macular degeneration, glaucoma, macular edema, chorioretinopathy, optic neuritis, ocular hypertension, optic neuropathy, or a combination thereof. In some cases, a user may implement multiple neuro-ophthalmic examinations described above, over a period of time. Across time, a user's condition may be monitored to determine if any corresponding neuro-ophthalmic conditions are improving or worsening.

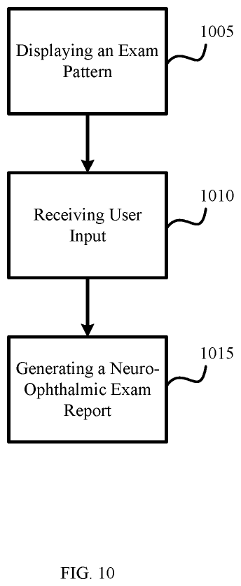

FIG. 10 depicts a process flow for generating a neuro-ophthalmic examination according to an embodiment of the present disclosure. The process flow can be implemented by system 100 of FIG. 1. In some cases, the process flow can be implemented by computing device 110 of FIG. 1.

At Step 1005, a neuro-ophthalmic examination pattern can be displayed. The neuro-ophthalmic examination can include a plurality of segments, where each of the plurality of segments is displayed at a time offset and at a discrete location of a display screen compared to other segments of the plurality of segments. The segments can be generated by object generator 205 of FIG. 2, for example.

At Step 1010, user input can be received. The user input can be received during a time period which a given segment of the plurality of segments is displayed on the display screen. In some cases, the user input can be received via computer mouse, a touchscreen, a microphone, a keyboard, a video camera, or a combination thereof. The input can be received a user input receiver 210 of FIG. 2.

At Step 1015, a neuro-ophthalmic examination report can be generated based on the displaying the neuro-ophthalmic examination pattern and the received user input. In some cases, the neuro-ophthalmic examination report comprises an assessment of a user's peripheral vision. In some cases, the neuro-ophthalmic examination report is indicative of macular degeneration, glaucoma, macular edema, chorioretinopathy, optic neuritis, ocular hypertension, optic neuropathy, or a combination thereof. The examination report can be generated by the report generator 220 of FIG. 2.

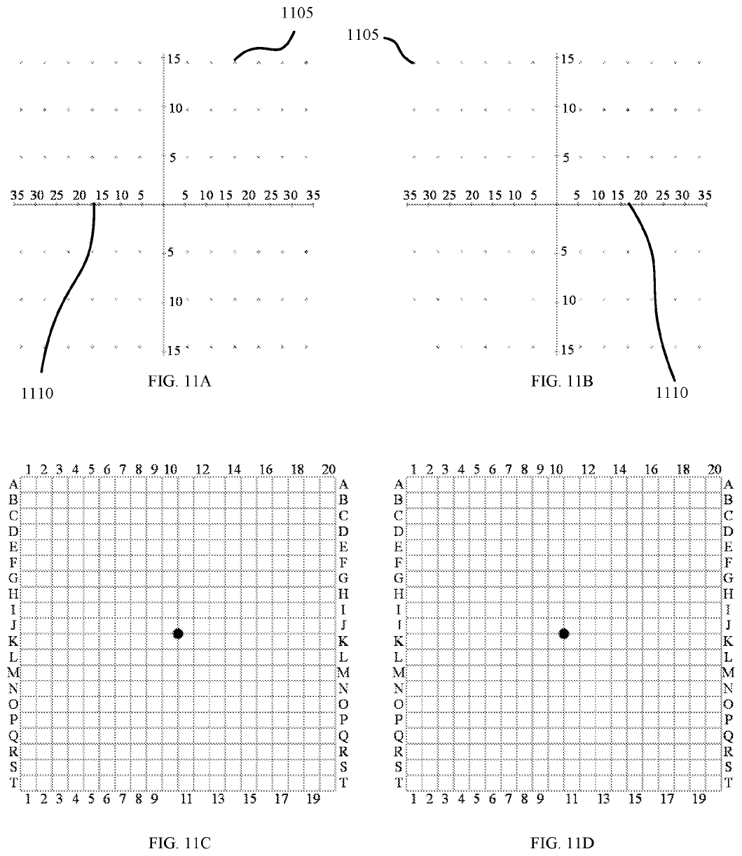

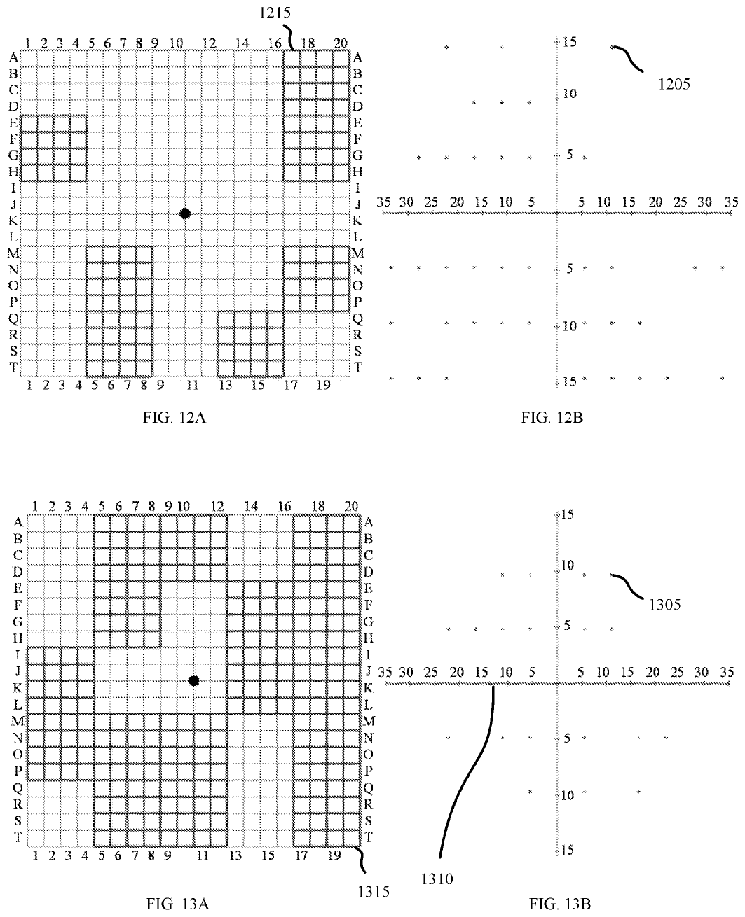

Referring now to FIGS. 11A-11D, 12A-12B, and 13A-13B, various visual segments of a neuro-ophthalmic examination of a study of patients are illustrated, according to an embodiment of the present disclosure. The study was conducted with 15 patients with an average age of 52.9 (±16.2) years old who have been diagnosed with brain tumors. The patients underwent a computerized Amsler grid assessment to evaluate for visual disturbances in their central vision. The Amsler grid assessment is designed to test 20 degrees of central vision. Each square of the grid was displayed in a sequential order; if the user notices a visual disturbance or abnormality, the patient was asked to press a key. The square at which a key was pressed is highlighted with bold lines (e.g., see FIG. 12A and FIG. 13A). The participants' performance on the computerized Amsler grid was compared to a computerized full visual field assessment (e.g., see FIG. 11A-11B). In the computerized full visual field assessment, participants are shown flashing dots at 5-degree intervals in all four quadrants of the participants' peripheral vision; participants are asked to press a key when they see the dot appears. If the participant sees the flashing dot and presses a key, a blue dot (e.g., dot 1105) is shown on the visual field chart. If the participant did not see the dot and did not press a key, the blue dot is missing from that location. It should be noted that the dots (e.g., dot 1105) illustrated throughout the drawings (e.g., FIGS. 11A-11B, FIG. 12B, and FIG. 13B) have various shades, indicating the shade at the time the patient pressed the button. Thus, a lighter shade dot indicates the patient pressed the button earlier than a darker shade dot. An absence of a dot indicates a patient was unable to detect a dot at all.

Three cases illustrated in FIGS. 11A-11D, 12A-12B, and 13A-13B are illustrated in connection with a plurality of patients. FIGS. 11A-11D illustrate “Case 1”—a patient without any visual complaints whose Amsler grid and visual field assessments were both normal. FIGS. 12A-12B illustrate “Case 2”—a participant with right sided visual disturbances. FIGS. 13A-13B illustrate “Case 3”—a participant with significant left eye visual disturbances that are detected on the Amsler grid and confirmed with the visual field assessment.

Referring specifically to FIGS. 11A-11D, “Case 1” (including “Case 1A” and “Case 1”) is illustrated. Case 1A and Case 1B examined an 82-year old female patient without any visual symptoms who underwent the computerized Amsler grid and visual field assessments. The patient had no visual complaints and had no deficits on either test. The patient pressed the key correctly when every dot 1105 appeared in each of the four quadrants on the visual field assessment (see Case 1A illustrated in FIGS. 11A-11B). FIG. 11A illustrates a visual field for the patient's left eye. FIG. 11B illustrates a visual field for the patient's right eye. On each of FIGS. 11A and 11B, an example dot 1105 and a peripheral vision indicator 1110 is illustrated. When the patient underwent the computerized Amsler grid, every square was visualized clearly without any abnormalities, therefore no bolded squares are shown (see Case 1B illustrated in FIGS. 11C-11D). FIG. 11C illustrates an Amsler grid for the patient's left eye. FIG. 11D illustrates an Amsler grid for the patient's right eye. Case 1 served as a negative control for the study.

Referring now to FIGS. 12A-12B, “Case 2” is illustrated. Case 2 examined a 35-year old male with visual disturbances in each of the four quadrants of the right eye, most pronounced in the right upper and right lower quadrants (e.g., upper and lower temporal quadrants), as illustrated by the plurality of bolded squares 1215 and the plurality (or lack thereof) of dots 1205 in FIGS. 12A-12B. FIG. 12A illustrates an Amsler grid for the patient's right eye. FIG. 12B illustrates a visual field for the patient's right eye.

Referring now to FIGS. 13A-13B, “Case 3” is illustrated. Case 3 examined a 75-year old man with severe visual disturbances in his left eye. FIG. 13A illustrates an Amsler grid for the patient's left eye. FIG. 13B illustrates a visual field for the patient's left eye. As illustrated in FIG. 13A, the computerized Amsler grid showed significant abnormalities in all four quadrants of his left eye field of view that correlated well with the missing dots in the visual field assessment grid. These findings are illustrated by the plurality of bolded squares 1315 and the plurality (or lack thereof) of dots 1305 in FIGS. 13A-13B. A peripheral vision indicator 1310 is illustrated, for reference.

### Enumerated Embodiments

The following enumerated embodiments are provided, the numbering of which is not to be construed as designating levels of importance.

Embodiment 1 provides a computer-implemented method including:
(a) displaying a neuro-ophthalmic examination pattern comprising a plurality of segments, wherein each of the plurality of segments is displayed at a time offset and at a discrete location of a display screen compared to other segments of the plurality of segments;
(b) receiving user input during a time period which a given segment of the plurality of segments is displayed on the display screen; and
(c) generating a neuro-ophthalmic examination report based on the displaying the neuro-ophthalmic examination pattern and the received user input.

Embodiment 2 provides the computer-implemented method of embodiment 1, wherein the neuro-ophthalmic examination pattern comprises an Amsler grid.

Embodiment 3 provides the computer-implemented method of any one of embodiments 1-2, wherein the user input is received via a computer mouse, a touchscreen, a microphone, a keyboard, a video camera, or a combination thereof.

Embodiment 4 provides the computer-implemented method of any one of embodiments 1-3, wherein the received user input is indicative of a distortion of the given segment when viewed by a user via the display screen.

Embodiment 5 provides the computer-implemented method of any one of embodiments 1-4, wherein the neuro-ophthalmic examination report comprises an assessment of a user's peripheral vision.

Embodiment 6 provides the computer-implemented method of embodiment 5, wherein the neuro-ophthalmic examination report is indicative of macular degeneration, glaucoma, macular edema, chorioretinopathy, optic neuritis, ocular hypertension, optic neuropathy, or a combination thereof.

Embodiment 7 provides the computer-implemented method of any one of embodiments 1-6, wherein each segment of the plurality of segments comprises a grid segment.

Embodiment 8 provides the computer-implemented method of any one of embodiments 1-7, wherein the displaying is implemented in a predetermined pattern.

Embodiment 9 provides the computer-implemented method of embodiment 8, wherein the predetermined pattern comprises a helical pattern beginning from a center point of the display screen and spanning outwardly.

Embodiment 10 provides the computer-implemented method of embodiment 8, wherein the predetermined pattern comprises a helical pattern beginning from an edge of the display screen and spanning inwardly.

Embodiment 11 provides the computer-implemented method of any one of embodiments 1-10, further including: repeating the steps of embodiment 1 (i.e., steps (a), (b), and (c)) with a second predetermined pattern for displaying the plurality of segments.

Embodiment 12 provides the computer-implemented method of any one of embodiments 1-11, wherein each of the plurality of segments is removed from the display screen after expiration of the respective time period.

Embodiment 13 provides the computer-implemented method of any one of embodiments 1-12, wherein each of the plurality of segments is continued to be displayed after expiration of the respective time period.

Embodiment 14 provides a device for generating a neuro-ophthalmic examination report, including:
a display screen;
a user input mechanism; and
one or more processors configured to execute a set of instructions that cause the one or more processors to: (a) display, via the display screen, a neuro-ophthalmic examination pattern comprising a plurality of segments, wherein each of the plurality of segments is displayed at a time offset and at a discrete location of a display screen compared to other segments of the plurality of segments; (b) receive, via the user input mechanism, user input during a time period which a given segment of the plurality of segments is displayed on the display screen; and (c) generate a neuro-ophthalmic examination report based on the displaying the neuro-ophthalmic examination pattern and the received user input.

Embodiment 15 provides a computer-readable medium for generating a neuro-ophthalmic examination report, including:
one or more processors;
memory; and
a set of instructions stored in the memory that, when executed by the one or more processors, cause the one or more processors to: (a) display, via a display screen, a neuro-ophthalmic examination pattern comprising a plurality of segments, wherein each of the plurality of segments is displayed at a time offset and at a discrete location of a display screen compared to other segments of the plurality of segments; (b) receive user input during a time period which a given segment of the plurality of segments is displayed on the display screen; and (c) generate a neuro-ophthalmic examination report based on the displaying the neuro-ophthalmic examination pattern and the received user input.

Embodiment 16 provides the device of embodiment 14, wherein the device is configured and adapted to implement any of the methods of embodiments 1-13.

Embodiment 17 provides the computer-readable medium of embodiment 15, wherein the computer-readable medium is configured and adapted to implement any of the methods of embodiments 1-13.

### EQUIVALENTS

Although preferred embodiments of the invention have been described using specific terms, such description is for illustrative purposes only, and it is to be understood that changes and variations may be made without departing from the spirit or scope of the following claims.

### INCORPORATION BY REFERENCE

The entire contents of all patents, published patent applications, and other references cited herein are hereby expressly incorporated herein in their entireties by reference.

1. A computer-implemented method comprising:
displaying a neuro-ophthalmic examination pattern comprising a plurality of segments, wherein each of the plurality of segments is displayed at a time offset and at a discrete location of a display screen compared to other segments of the plurality of segments;
receiving user input during a time period which a given segment of the plurality of segments is displayed on the display screen; and
generating a neuro-ophthalmic examination report based on the displaying the neuro-ophthalmic examination pattern and the received user input.

2. The computer-implemented method of claim 1, wherein the neuro-ophthalmic examination pattern comprises an Amsler grid.

3. The computer-implemented method of claim 1, wherein the user input is received via a computer mouse, a touchscreen, a microphone, a keyboard, a video camera, or a combination thereof.

4. The computer-implemented method of claim 1, wherein the received user input is indicative of a distortion of the given segment when viewed by a user via the display screen.

5. The computer-implemented method of claim 1, wherein the neuro-ophthalmic examination report comprises an assessment of a user's peripheral vision.

6. The computer-implemented method of claim 5, wherein the neuro-ophthalmic examination report is indicative of macular degeneration, glaucoma, macular edema, chorioretinopathy, optic neuritis, ocular hypertension, optic neuropathy, or a combination thereof.

7. The computer-implemented method of claim 1, wherein each segment of the plurality of segments comprises a grid segment.

8. The computer-implemented method of claim 1, wherein the displaying is implemented in a predetermined pattern.

9. The computer-implemented method of claim 8, wherein the predetermined pattern comprises a helical pattern beginning from a center point of the display screen and spanning outwardly.

10. The computer-implemented method of claim 8, wherein the predetermined pattern comprises a helical pattern beginning from an edge of the display screen and spanning inwardly.

11. The computer-implemented method of claim 1, further comprising:
repeating the steps of claim 1 with a second predetermined pattern for displaying the plurality of segments.

12. The computer-implemented method of claim 1, wherein each of the plurality of segments is removed from the display screen after expiration of the respective time period.

13. The computer-implemented method of claim 1, wherein each of the plurality of segments is continued to be displayed after expiration of the respective time period.

14. A device for generating a neuro-ophthalmic examination report, comprising:
a display screen;
a user input mechanism; and
one or more processors configured to execute a set of instructions that cause the one or more processors to:
display, via the display screen, a neuro-ophthalmic examination pattern comprising a plurality of segments, wherein each of the plurality of segments is displayed at a time offset and at a discrete location of a display screen compared to other segments of the plurality of segments;
receive, via the user input mechanism, user input during a time period which a given segment of the plurality of segments is displayed on the display screen; and
generate a neuro-ophthalmic examination report based on the displaying the neuro-ophthalmic examination pattern and the received user input.

15. A computer-readable medium for generating a neuro-ophthalmic examination report, comprising:
one or more processors;
memory; and
a set of instructions stored in the memory that, when executed by the one or more processors, cause the one or more processors to:
display, via a display screen, a neuro-ophthalmic examination pattern comprising a plurality of segments, wherein each of the plurality of segments is displayed at a time offset and at a discrete location of a display screen compared to other segments of the plurality of segments;
receive user input during a time period which a given segment of the plurality of segments is displayed on the display screen; and
generate a neuro-ophthalmic examination report based on the displaying the neuro-ophthalmic examination pattern and the received user input.
